(19) United States
(12) Patent Application Publication
(10) Pub. No.: US 2026/0224194 A1
(43) Pub. Date: Aug. 6, 2026

(54) FORWARD- AND SIDE-VIEWING INTRACARDIAC ECHOCARDIOGRAPY CATHETER AND SYSTEM

(71) Applicant: Boston Scientific Scimed, Inc., Maple Grove, MN (US)

(72) Inventors: Binh C. Tran, St. Paul, MN (US); Daniel J. Foster, Lino Lakes, MN (US)

(21) Appl. No.: 19/465,833

(22) Filed: Jan. 30, 2026

Related U.S. Application Data
(60) Provisional application No. 63/752,537, filed on Jan. 31, 2025.

(51) Int. Cl. A61B 8/12; A61B 8/00; A61B 8/08; G06T 7/60

(52) U.S. Cl. CPC A61B 8/12; A61B 8/0883; A61B 8/4488; A61B 8/4494; A61B 8/488; A61B 8/5223; G06T 7/60; A61B 2560/0462; A61B 2562/0204; A61B 2562/028; A61B 2562/046; G06T 2207/10132; G06T 2207/30048

(57) ABSTRACT

A medical imaging catheter to be extended into a patient’s heart is disclosed. The medical imaging catheter includes a shaft having a proximal region and an opposite distal region. The shaft defines a longitudinal axis. An imaging module is coupled to the distal region to be extended into the patient’s heart. The imaging module includes a side viewing ultrasound imaging device having a planar active surface to image in a direction perpendicular to the longitudinal axis. The imaging module also includes a front viewing imaging device to image in a direction along the longitudinal axis.

### CROSS REFERENCE TO RELATED APPLICATIONS

This application claims priority to U.S. Provisional Application No. 63/752,537 entitled “FORWARD- AND SIDE-VIEWING INTRACARDIAC ECHOCARDIOGRAPHY CATHETER AND SYSTEM,” filed January 31, 2025, which is hereby incorporated by reference in its entirety.

### TECHNICAL FIELD

The present disclosure relates to medical systems and methods for facilitating visualization of tissue from within a patient. More specifically, the present disclosure relates to medical systems, catheters, and methods having ultrasonic transducer devices.

### BACKGROUND

Intracardiac echocardiography (ICE) is an ultrasonic imaging modality that has become a common aspect of several percutaneous interventional and electrophysiology procedures. ICE can provide high resolution and real-time visualizations of cardiac structures, continuous monitoring of catheter location within the heart, and early recognition of procedural complications such as pericardial effusion or thrombus formation. An ICE catheter is typically inserted in a vein, such as via standard femoral venous introducer into the femoral vein, and then passed from the vein into the heart alongside other tools. Another common ultrasonic imaging modality includes transesophageal echocardiography (TEE) and fluoroscopy to image and visualize tools within the heart, which requires an anesthesiologist and additional echocardiologist to keep the patient under general anesthesia and to manipulate an ultrasonic probe within the airway. In contrast to TEE, ICE can be performed by the primary operator of the interventional procedure under conscious sedation, without endotracheal intubation, and with a reduced risk of esophageal trauma. Additionally, ICE reduces fluoroscopy exposure for both the patient and the operator. For these reasons, ICE is a preferred imaging modality in certain procedures such as atrial septal defect closure and catheter ablation of cardiac arrhythmias and includes an emerging role in other procedures such as mitral valvuloplasty, transcatheter aortic valve replacement, and left atrial appendage closure.

Two forms of ICE are available. Radial or rotational ICE uses a single piezoelectric crystal mounted on a tip of a six to ten French catheter. A rotating transducer provides cross-sectional images in a radial plane perpendicular to a longitudinal axis of the catheter. Rotational ICE operates at imaging frequencies that are useful for near-field imaging of up to six or eight centimeters but is limited for far-field imaging. Phased-array ICE uses a multi-element transducer, such as a sixty-four-element transducer, mounted on a distal end of an eight to ten French steerable catheter that can often be deflected in four directions including anterior, posterior, right, and left. The device provides a wedge-shaped image, such as a ninety-degree sector plane, from the side of the catheter that can be displayed on a conventional ultrasound workstation. When contrasted with mechanical rotational ICE systems, phased-array ICE can provide for greater image-depth penetration (up to 15 cm), greater maneuverability, and the ability to acquire Doppler and color flow imaging.

### SUMMARY

In Example 1, a medical imaging catheter configured to be extended into a patient’s heart, the medical imaging catheter comprising: a shaft having a proximal region and an opposite distal region, the shaft defining a longitudinal axis; and an imaging module coupled to the distal region and configured to be extended into the patient’s heart, the imaging module including: a side viewing ultrasound imaging device having a planar active surface, the planar active surface configured to image in a direction perpendicular to the longitudinal axis; and a front viewing imaging device configured to image in a direction along the longitudinal axis.

In Example 2, the medical imaging catheter of Example 1, wherein the medical imaging catheter is an intracardiac echocardiography (ICE) device.

In Example 3, the medical imaging catheter of any of Examples 1 and 2, wherein the side viewing ultrasound imaging device includes a micro-electromechanical system (MEMS) transducer array.

In Example 4, the medical imaging catheter of Example 3, wherein the MEMS transducer array includes a piezoelectric micro-machined ultrasound transducer (pMUT).

In Example 5, the medical imaging catheter of any of Examples 1-4, wherein the front viewing imaging device is configured to generate an optical signal in the direction along the longitudinal axis.

In Example 6, the medical imaging catheter of Example 5, wherein the front viewing imaging device includes an optical fiber.

In Example 7, the medical imaging catheter of any of Examples 5 and 6, and further comprising a medical imaging console coupled to the medical imaging catheter, the medical imaging console including a controller having a time-of-flight measurement circuit configured to measure a time-of-flight of the optical signal.

In Example 8, the medical imaging catheter of Example 7, wherein the controller is configured to detect an aperture in a septum of the heart or proximity to a wall of the heart based on the measured time-of-flight.

In Example 9, the medical imaging catheter of any of Example 1-4, wherein the front viewing imaging device includes an ultrasound transducer.

In Example 10, the medical imaging catheter of Example 9, wherein the ultrasound transducer of front viewing imaging device includes a pMUT.

In Example 11, the medical imaging catheter of any of Examples 9 and 10, and further comprising a flexible circuit substrate, wherein the side viewing ultrasound imaging device and the ultrasound transducer of the front viewing imaging device are disposed on the flexible circuit substrate.

In Example 12, the medical imaging catheter of Example 11, wherein the flexible circuit substrate includes a first planar assembly segment having the side viewing ultrasound device and a second planar assembly segment having the ultrasound transducer of the front viewing imaging device, wherein the second planar assembly segment is bent to be perpendicular to the first planar assembly segment.

In Example 13, the medical imaging catheter of any of Examples 9-12, and further comprising a medical imaging console coupled to the medical imaging catheter, the medical imaging console including a controller configured to measure depth or distance of the image in the direction of the longitudinal axis and detect an aperture in a septum of the heart or proximity to a wall of the heart based on the depth of distance of the image.

In Example 14, the medical imaging catheter of any of Examples 9-12, and further comprising a medical imaging console coupled to the medical imaging catheter, the medical imaging console including a controller configured to Doppler effects of the of the image in the direction of the longitudinal axis and detect an aperture in a septum of the heart based on the Doppler effects of the image.

In Example 15, the medical imaging catheter of any of Examples 13 and 14, wherein the controller is configured to generate an alert if the aperture in the septum is detected or proximity to a wall of the heart is closer than a threshold amount.

In Example 16, a medical imaging catheter configured to be extended into a patient’s heart, the medical imaging catheter comprising: a shaft having a proximal region and an opposite distal region, the shaft defining a longitudinal axis; and an imaging module coupled to the distal region and configured to be extended into the patient’s heart, the imaging module including: a side viewing ultrasound imaging device having a planar active surface, the planar active surface configured to image in a direction perpendicular to the longitudinal axis; and a front viewing imaging device configured to image in a direction along the longitudinal axis.

In Example 17, the medical imaging catheter of Example 16, wherein the medical imaging catheter is an intracardiac echocardiography (ICE) device.

In Example 18, the medical imaging catheter of Example 16, wherein the side viewing ultrasound imaging device includes a micro-electromechanical system (MEMS) transducer array.

In Example 19, the medical imaging catheter of Example 18, wherein the MEMS transducer array includes a piezoelectric micro-machined ultrasound transducer (pMUT).

In Example 20, the medical imaging catheter of Example 16, wherein the front viewing imaging device is configured to generate an optical signal in the direction along the longitudinal axis.

In Example 21, the medical imaging catheter of Example 20, wherein the front viewing imaging device includes an optical fiber.

In Example 22, the medical imaging catheter of Example 20, and further comprising a medical imaging console coupled to the medical imaging catheter, the medical imaging console including a controller having a time-of-flight measurement circuit configured to measure a time-of-flight of the optical signal.

In Example 23, the medical imaging catheter of Example 22, wherein the controller is configured to detect an aperture in a septum of the heart or proximity to a wall of the heart based on the measured time-of-flight.

In Example 24, the medical imaging catheter of Example 23, wherein the front viewing imaging device includes an ultrasound transducer.

In Example 25, the medical imaging catheter of Example 24, wherein the ultrasound transducer of front viewing imaging device includes a pMUT.

In Example 26, the medical imaging catheter of Example 23, and further comprising a flexible circuit substrate, wherein the side viewing ultrasound imaging device and the ultrasound transducer of the front viewing imaging device are disposed on the flexible circuit substrate.

In Example 27, the medical imaging catheter of Example 26, wherein the flexible circuit substrate includes a first planar assembly segment having the side viewing ultrasound device and a second planar assembly segment having the ultrasound transducer of the front viewing imaging device, wherein the second planar assembly segment is bent to be perpendicular to the first planar assembly segment.

In Example 28, the medical imaging catheter of Example 24, and further comprising a medical imaging console coupled to the medical imaging catheter, the medical imaging console including a controller configured to measure depth or distance of the image in the direction of the longitudinal axis and detect an aperture in a septum of the heart or proximity to a wall of the heart based on the depth of distance of the image.

In Example 29, the medical imaging catheter of Example 28, wherein the controller is configured to generate an alert if the aperture in the septum is detected or proximity to a wall of the heart is closer than a threshold amount.

In Example 30, the medical imaging catheter of Example 24, and further comprising a medical imaging console coupled to the medical imaging catheter, the medical imaging console including a controller configured to Doppler effects of the of the image in the direction of the longitudinal axis and detect an aperture in a septum of the heart based on the Doppler effects of the image.

In Example 31, an intracardiac echocardiography (ICE) catheter configured to be extended into a patient’s heart, the medical imaging catheter comprising: a shaft having a proximal region and an opposite distal region, the shaft defining a longitudinal axis; and an imaging module coupled to the distal region and configured to be extended into the patient’s heart, the imaging module including: a side viewing ultrasound imaging device having a planar active surface, the planar active surface configured to image in a direction perpendicular to the longitudinal axis; a front viewing imaging device configured to image in a direction along the longitudinal axis, wherein the front viewing imaging device includes an ultrasound transducer; and a flexible circuit substrate, wherein the side viewing ultrasound imaging device and the ultrasound transducer of the front viewing imaging device are disposed on the flexible circuit substrate, wherein the flexible circuit substrate includes a first planar assembly segment having the side viewing ultrasound device and a second planar assembly segment having the ultrasound transducer of the front viewing imaging device, wherein the second planar assembly segment is bent to be perpendicular to the first planar assembly segment.

In Example 32, the ICE catheter of Example 31, wherein the side viewing ultrasound imaging device includes a micro-electromechanical system (MEMS) transducer array and the front viewing imaging device includes a MEMS transducer array.

In Example 33, the ICE catheter of Example 32, wherein each of the MEMS transducer arrays includes a piezoelectric micro-machined ultrasound transducer (pMUT).

In Example 34, a method for use with a medical imaging system having a medical imaging catheter configured to be extended into a patient’s heart, the medical imaging catheter comprising an imaging module coupled to a distal region of a shaft and configured to be extended into the patient’s heart, the imaging module including a side viewing ultrasound imaging device having a planar active surface, the planar active surface configured to image in a direction perpendicular to a longitudinal axis of the shaft, and a front viewing imaging device configured to image in a direction along the longitudinal axis, the method comprising at least one of: measuring depth or distance of the image in the direction of the longitudinal axis and detecting an aperture in a septum of the heart or proximity to a wall of the heart based on the depth of distance of the image; or applying Doppler effects of the of the image in the direction of the longitudinal axis and detecting an aperture in a septum of the heart based on the Doppler effects of the image..

In Example 35, the method of Example 34, comprising generating an alert if the aperture in the septum is detected or if proximity to a wall of the heart is closer than a threshold amount.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram a medical imaging system used while treating a patient such as treating a heart of the patient.

FIG. 2 is a schematic diagram of a region of a medical imaging catheter of the medical imaging system of FIG. 1.

FIG. 2A is a schematic diagram of a first embodiment of the region of the medical imaging catheter of FIG. 2

FIG. 2B is a schematic diagram of a second embodiment of the region of the medical imaging catheter of FIG. 2.

FIG. 2C is a schematic diagram of a third embodiment of the region of the medical imaging catheter of FIG. 2.

FIG. 3 is a schematic diagram of an ultrasound transducer assembly for use in a medical imaging catheter of the medical imaging system of FIG. 1.

FIG. 3A is a schematic diagram of a first embodiment of the ultrasound transducer assembly of FIG. 3 adapted for use in the medical imaging catheter of the medical imaging system of FIG. 1.

FIG. 3B is a schematic diagram of a second embodiment of the ultrasound transducer assembly of FIG. 3 adapted for use in the medical imaging catheter of the medical imaging system of FIG. 1.

FIG. 4 is a schematic diagram of a controller of a medical imaging console of the medical imaging system of FIG. 1.

FIG. 5 is a diagram of a partially sectioned heart with the region of the medical imaging catheter in a right atrium of the heart and an embodiment of a visualization generated with the controller of FIG. 4.

FIG. 6 is a schematic diagram of the partially section heart of FIG. 5 with the region of the medical imaging catheter in a left atrium of the heart and another embodiment of a visualization generated with the controller of FIG. 4.

FIG. 7 is a block diagram of a method of the controller of FIG. 4.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

Typical medical imaging catheter systems using intracardiac echocardiography (ICE) devices incorporate ultrasound transducers positioned along the shaft of the distal end of the catheter to provide side-viewing planar or volume imaging. For instance, side-viewing ICE devices produce a wedge-shaped image, such as a ninety-degree sector plane, which is plus/minus forty-five degrees in the axis perpendicular to the longitudinal axis of the distal end of the medical imaging catheter. In such a configuration, typical medical imaging catheter systems are not able to generate an image of what is in front of, i.e., in the direction of the longitudinal axis beyond the distal end, of the medical imaging catheter.

The lack of imaging in front of the catheter can lead to difficulties maneuvering the catheter to intended anatomical targets or procedural complications. In one example, ICE devices apply an approximately 10 MHz ultrasound signal, which includes a limited penetration depth that produces suboptimal quality of images of objects at a relatively larger distance. Image quality is improved when the ultrasound transducer is placed closed to the anatomy of interest, but safety concerns prevent many clinicians from attempting to maneuver the catheter closer to target anatomy. In one example, a side-viewing ICE device is not able to see a puncture in the fossa ovalis to cross the septum from the right atrium to the left atrium as the clinician attempts to thread the tip of the catheter through the puncture site. Once inside the left atrium, the clinician images the pulmonary veins without being aware whether the tip of the catheter is pressing against the atrium wall.

The disclosure provides a medical imaging system and medical imaging catheter, such as an ICE device, incorporating a side viewing ultrasound transducer and a front viewing imaging device. The medical imaging system and medical imaging catheter with side-viewing and front-viewing capabilities can improve maneuvering to intended anatomical targets, reduce potential complications, and provide additional capabilities such as tissue thickness measurements and alerts. In some embodiments, the front viewing imaging device on the medical imaging catheter is an optical or fiber optic sensor. In some embodiments, the front viewing imaging device is an ultrasound transducer. In some embodiments, the front viewing ultrasound transducer produces A-mode or M-mode imaging, such as a single element ultrasound transducer. In one embodiment, the front viewing ultrasound transducer can operate with a center frequency in the range of 50 MHz to 75 MHz to produce relatively high resolution close to the front viewing ultrasound transducer with relatively limited dept and field view. In another embodiment, the front viewing ultrasound transducer can operate with a center frequency in the range of 7.5 MHz to 20 MHz to produce relatively moderate resolution with relatively moderate depth penetration and field of view. In some embodiments, the front viewing ultrasound transducer is a multielement ultrasound transducer configured for planar or volume imaging. The medical imaging system, in some embodiments, is configured to detect anatomical openings in front of the catheter tip to facilitate navigation of the catheter through the opening. In some embodiments, the medical imaging system employs delayed return echo techniques to detect the opening. For example, the echo delay will increase when the catheter tip is pointed at an opening rather than a proximate tissue boundary. In some embodiments, the medical imaging system employs doppler techniques to detect the opening. For example, the doppler flow will be increased when the tip is pointed at a vessel or a tissue opening versus when the tip is pointed at a tissue boundary. In some embodiments, the medical imaging system provides a first alert when the catheter tip is pointed at an opening and a second alert when the catheter tip is proximate a tissue boundary, which is useful when a clinician is attempting to cross through the septum from the right atrium to the left atrium and when the clinician is viewing the pulmonary veins to detect the proximity of the catheter tip to the left atrial wall. In some embodiments, the medical imaging system can apply the front viewing device to measure tissue thickness.

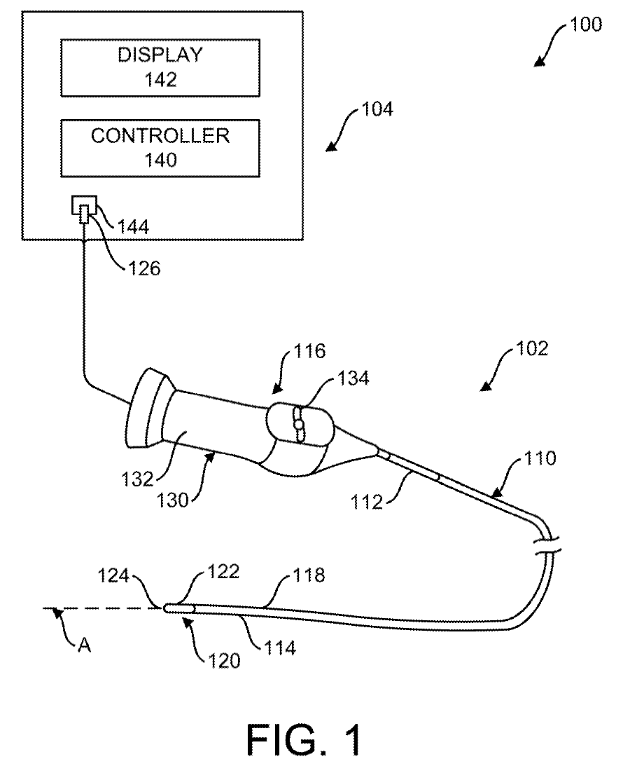

FIG. 1 illustrates an embodiment of a medical imaging system 100 suitable for use in a clinical setting for a medical procedure on a patient, such as a medical procedure, including an electrophysiology procedure or intracardiac procedure, on a heart of the patient. The medical imaging system 100 includes a medical imaging catheter system 102 configured to be inserted into the patient for imaging of portions of the patient’s anatomy and a medical imaging console 104 configured to be coupled to the medical imaging catheter system 102 and generate an image on a device, such as a display. The medical imaging catheter system 100 includes a medical imaging catheter 110, an introducer sheath, and various connecting elements, such as cables and tubing, that connect the components of the medical imaging catheter system 102 to one another and to the components of the medical imaging console 104. The medical imaging system can be employed in transseptal catheterization for several percutaneous interventions, including left heart catheter ablation, atrial septal defect closure for effective alternative to surgical intervention. Access to the patient’s heart can be obtained through a vessel, such as a peripheral vein often in the groin, such as the femoral vein, or possibly in the shoulder or neck. Once access to the vessel is obtained, the medical imaging catheter 110 can be navigated to within the patient’s heart, such as within a chamber of the heart.

The medical imaging catheter 110 includes a proximal end region 112 and a distal end region 114. The medical imaging catheter 110 defines a longitudinal axis A that passes through a centroid of a cross section of the distal end region 114. The proximal end region 112 of the medical imaging catheter 110 includes a catheter hub 116, and the distal end region 114 is configured and arranged to be inserted into the patient. The medical imaging catheter 110 includes a shaft 118 that is dimensioned to be inserted within regions of the patient that are difficult to navigate, including blood vessels, heart chambers, and, in some cases, the gastrointestinal tract, the urinary tract, and the pulmonary system.

In embodiments, the medical imaging catheter 110 is configured as an ICE device. The distal end region 114 includes an imaging module 120 that can be, for example, extended into the patient’s heart and disposed at the end of the shaft 118. In the illustrated embodiment, the imaging module 120 can include an imaging module cover 122 forming a distalmost tip 124, the imaging module cover 122 being mechanically coupled to a distal end region 114 of the shaft 118. The imaging module includes an imaging transducer configured for imaging the patient’s anatomy such as target anatomy proximate the distal end region 114. In embodiments, the imaging module 120 includes an ultrasound imaging device configured to transmit and receive acoustic energy to generate ultrasound images of the target anatomy. The imaging module 120, such as the imaging transducer, is coupled to elongated lead conductors that extend from the distal end region 114 along the catheter shaft 118 to the proximal end region 112, such as to a plug 126 at the proximal end region 112. In one embodiment, the lead conductors include electrically conductive elements, configured to carry an electrical signal, such as wires or traces that are electrically insulated from one another within an insulative sheath, such as with an insulative polymer sheath, or disposed on a flex circuit extending the length of the shaft 118. In another embodiment, the lead conductors include optical conductors, configured to carry an optical signal, such as a flexible glass or plastic fiber optic cable in addition to or instead of the electrically conductive elements. The plug 126 is configured to be mechanically and electrically or optically coupled to the medical imaging console 104, for example, either directly or via intermediary cabling.

In the illustrated embodiment, the hub 116 includes a housing 130 forming handle assembly 132 and a steering control unit with a steering actuator 134. The steering control unit is provided for articulating the distal end region 114 and positioning the imaging module 120. The steering control unit includes, in some embodiments, steering cables coupled to the steering actuator 134 and disposed within the shaft 118 extending to the distal end region 114. The steering actuator 134 can be rotated to facilitate positioning of the imaging module 120. When the clinician rotates the steering actuator 134, a steering cable pulls the distal end region to bend the shaft 118 to select a position of the imaging module with respect to the target anatomy.

The medical imaging console 104 includes a controller 140, such one or more controllers, processors, or computers, that executes instructions or code, such as processor-executable instructions, stored on a non-transitory computer readable medium, such as a memory device, or memory, to cause, such as control or perform, the aspects of the medical imaging catheter system 100. The medical imaging console 104, includes a receptacle 144 configured to couple to cabling or plug 126 and a display 142, or is configured to be coupled to a display 142 via a display connection. The controller 140 is configured to provide a signal, such as a plurality of concurrent or space-apart-time electrical signals, to the connected medical imaging catheter 110 via the receptacle 142 that is transmitted along the lead conductors to the imaging module 120. The controller 140 is also configured to receive a signal, such as a plurality of concurrent or space-apart-time electrical or optical signals, from the connected medical imaging catheter 110 along lead conductors from the imaging module 120. The imaging module 120 includes an ultrasound imaging device configured to transmit and receive acoustic energy to generate ultrasound images with the medical imaging console 104.

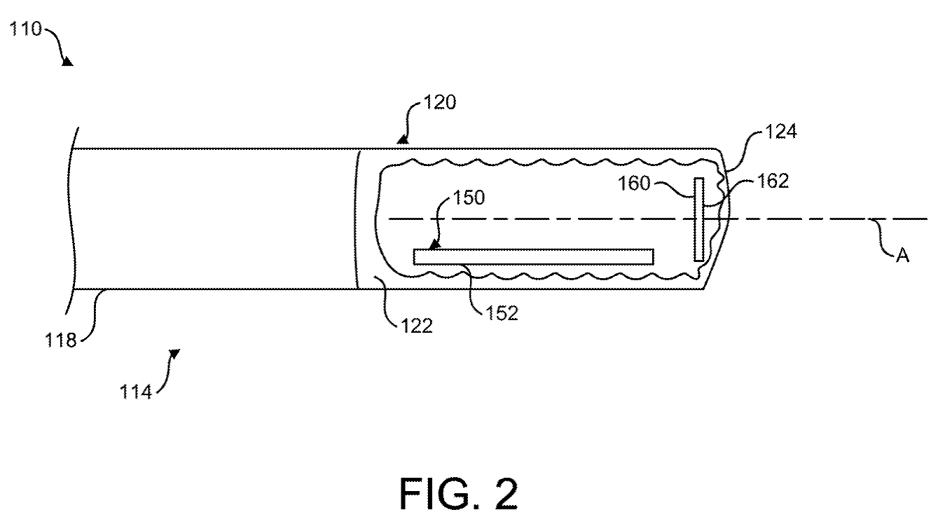

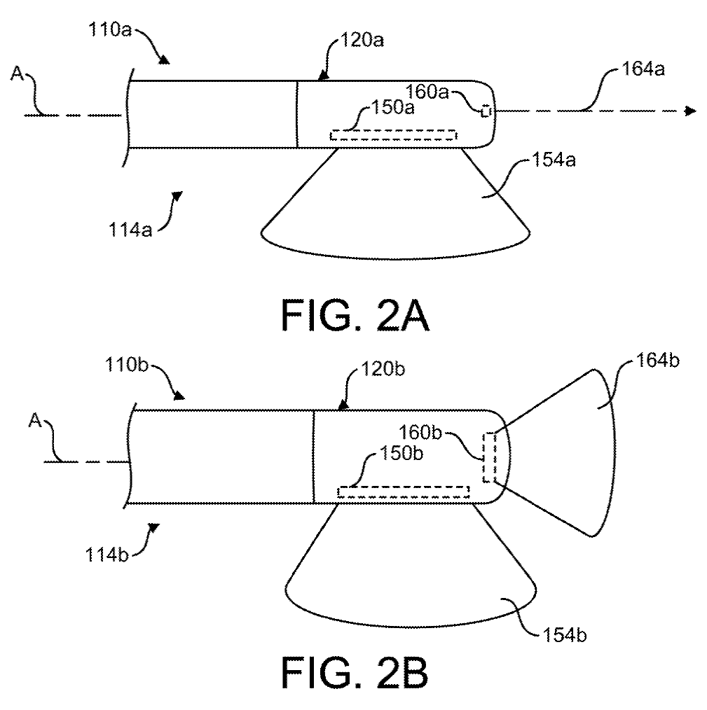

FIG. 2 illustrates an embodiment of the distal end region 114 of the medical imaging catheter 110, which includes the imaging module 120 that can be extended into the patient’s heart and disposed at the end of a shaft 118. In the illustrated embodiment, the imaging module 120 include the imaging module cover 122 forming the distalmost tip 124, although other embodiments are contemplated. The imaging module 120 also includes a side viewing ultrasound imaging device 150 and a front viewing imaging device 160. The cover 122 encloses the side and front viewing imaging devices 150, 160. The cover 122 is partially cut away to illustrate the side and front viewing devices 150, 160 inside the imaging module 120. The cover 122 can be constructed from a material having electrically insulative and low-loss acoustic properties to form an acoustic window, such as an epoxy encapsulant or a polyether block amide (PEBA) such as available under the trade designation PEBAX or a thermoplastic elastomer (TPE) available under the trade name VISTAMID. The side viewing ultrasound imaging device 150 is disposed within the cover 122 with respect to the longitudinal axis A in such a manner to produce a first, or side, image perpendicular to the longitudinal axis A, and the front viewing imaging device 160 is disposed within the cover 122 with respect to the longitudinal axis A in such a manner to produce a second, or front, image in the direction of the longitudinal axis A. In the illustrated embodiment, the side viewing ultrasound imaging device 150 includes an operable or active surface 152 configured for producing an acoustic signal and receiving echoes that is aligned parallel with the longitudinal axis A and configured to image in a direction perpendicular to the longitudinal axis A. The front viewing imaging device 160 includes an operable or active surface 162 configured to produce an acoustic signal and receive echoes or receive an optical signal that is aligned perpendicular to the longitudinal axis A and perpendicular to the operable or active surface 152 of the side viewing ultrasound imaging device 150. The operable or active surface 162 of the front viewing imaging device 160 is configured to image in a direction along the longitudinal axis A.

The side viewing ultrasound imaging device 150, in some embodiments, provides a wedge-shaped image, such as a ninety-degree sector plane, from the side of the distal end of the medical imaging catheter 110 that can be displayed on the medical imaging console 104. In one example of the side viewing ultrasound imaging device 150, a two-dimensional imaging phased array device, which include one-dimensional arrays, creates two-dimensional images by steering an acoustic beam across a two-dimensional plane. Each acoustic beam, or scan line, produces echoes that are measured and combined with other scan lines into an ultrasound image. To create a three-dimensional or four-dimensional (including time as a dimension) image, a two-dimensional array is applied to steer the beam throughout a three-dimensional volume or two orthogonally disposed one-dimensional arrays scan each scan beams across a respective two-dimensional plane. The side viewing ultrasound imaging device 150 still produces a wedge-shaped image, such as a ninety-degree sector plane, which is plus/minus forty-five degrees in the axis perpendicular to the longitudinal axis A. In such a configuration, the medical imaging catheter 110 is not able to generate an image of what is in front of, i.e., in the direction of the longitudinal axis beyond the distal end, of the medical imaging catheter 110.

In one embodiment, the front viewing imaging device 160 is an ultrasound transducer configured with respect to the longitudinal axis A to produce acoustic energy as a focused beam in the direction along the longitudinal axis A in front of the distalmost tip 124 and to receive echoes in the direction along the longitudinal axis A from in front of the distalmost tip 124. In one embodiment, the ultrasound transducer of the front viewing imaging device 160 operates with a center frequency in the range of 50 MHz to 75 MHz to produce relatively high resolution close to the distalmost tip 124 with relatively limited dept and field view. In another embodiment, the ultrasound transducer of the front viewing imaging device 160 operates with a center frequency in the range of 7.5 MHz to 20 MHz to produce relatively moderate resolution with relatively moderate depth penetration and field of view. In some embodiments, the ultrasound transducer of the front viewing imaging device 160 is a single element ultrasound transducer configured to provide amplitude mode, or A-mode, imaging. The controller 140 of the medical imaging console 104 can determine information from the A-mode imaging such as distance along the longitudinal axis of the ultrasound transducer or distalmost tip 124 to tissue or the thickness of tissue in front of the catheter 110 along the longitudinal axis. In some embodiments, the ultrasound transducer of the front viewing imaging device 160 is a multielement ultrasound transducer configured for planar or volume imaging. The multielement ultrasound transducer of the front viewing imaging device 160 produces a wedge-shaped image, such as a ninety-degree sector plane, which is plus/minus forty-five degrees to the longitudinal axis A.

FIGS. 2A and 2B illustrate embodiments of the distal end region 114a, 114b of the medical imaging catheter 110a, 110b, respectively, in which the front viewing imaging devices 160a, 160b are ultrasound transducers. FIG. 2A illustrates distal end region 114a having an imaging module 120a including a side viewing ultrasound imaging device 150a configured to produce an image perpendicular to the longitudinal axis and front viewing ultrasound imaging device 160a configured to produce an image in the direction of, or along, the longitudinal axis. In the illustrated example, the side viewing ultrasound imaging device 150a includes a multielement ultrasound transducer, and the front viewing ultrasound imaging device 160a includes a single element ultrasound transducer. The side viewing ultrasound imaging device 150a is configured for planar or volume imaging over time, as indicated with sector plane 154a. The front viewing ultrasound imaging device 160a is configured for an amplitude trace over time, as indicated with arrow 164a. In one embodiment, the single element ultrasound transducer of the front viewing ultrasound imaging device 160a is configured to operate in the 20-50 MHz range and provide range-finding and depth detection functionality. FIG. 2B illustrates distal end region 114b having an imaging module 120b including a side viewing ultrasound imaging device 150b configured to produce an image perpendicular to the longitudinal axis and front viewing ultrasound imaging device 160b configured to produce an image in the direction of the longitudinal axis. In the illustrated example, both the side viewing ultrasound imaging device 150b and front viewing ultrasound imaging device 160b include a multielement ultrasound transducer. The side viewing ultrasound imaging device 150b and front viewing ultrasound imaging device 160b are configured for planar or volume imaging over time, as indicated with sector planes 154b, 164b, respectively. In one embodiment, the front viewing ultrasound imaging device 160b includes a one dimensional or two-dimensional array to provide planar or volume imaging. To reduce the cross-sectional area of the catheter 110b and improve the tolerance and maneuverability of the catheter 110b, the front viewing ultrasound imaging device 160b is smaller in size, such as area of the operable surface, than the size of the side viewing ultrasound imaging device 150b.

FIG. 2C illustrates an embodiment of the distal end region 114c of the medical imaging catheter 110c in which the front viewing imaging device 160c is a device other than ultrasound transducer, such as an optical transducer or an optical fiber coupled to an optical transducer. FIG. 2C illustrates distal end region 114c having an imaging module 120c including a side viewing ultrasound imaging device 150c configured to produce an image perpendicular to the longitudinal axis and front viewing optical device 160c configured to produce an image or detect an optical signal in the direction of, or along, the longitudinal axis. In the illustrated example, the side viewing ultrasound imaging device 150c includes a multielement ultrasound transducer, and the front viewing optical imaging device 160c includes an optical transducer or optical fiber having a distal end coupled to the distalmost tip 124c. The side viewing ultrasound imaging device 150c is configured for planar or volume imaging over time, as indicated with sector plane 154c. In one embodiment, the front viewing optical imaging device 160c is configured for a measurement such as a time-of-flight measurement over time, like the amplitude trace over time, as indicated with arrow 164c. In one embodiment of an optical transducer as the front viewing optical device 160c in the imaging module 120c, the optical transducer receives an electrical signal and generates an optical signal 164c to emit along the longitudinal axis. In one embodiment of an optical fiber having a distal end, the optical fiber receives an optical signal, such as from the medical imaging console 104, and transmits the optical signal along the shaft 118c to the distal end of the optical fiber, where the optical signal 164c emits along the longitudinal axis. The front viewing optical device 160c is also configured to receive a reflection of the optical signal. In some embodiments, the imaging module 120c includes a plurality of optical fibers, such as a transmit optical fiber to provide an optical signal along the direction of the longitudinal axis and a receive optical fiber to receive reflections of the optical signal. In some embodiments, a single optical fiber can perform the functions of a transmit and receive optical fibers.

In one embodiment of the medical imaging catheter 110c with an optical transducer or optical fiber as the front viewing imaging device 160c, the medical imaging system 100 is configured to measure time of flight. Time of flight is measurement of the time taken by an object, particle, or wave (such as an acoustic wave or electromagnetic wave) to travel a distance through a medium. This information can be used to measure velocity or path length, or to learn about properties of the medium. In one embodiment, the travel distance is measured as a round trip distance from transducer through the optical fiber, through the medium, reflected off tissue within the heart, and returned through the medium to the optical fiber and back to the transducer. In another example, the travel distance is the travel from the tip of the optical fiber through the medium, reflected off of tissue and back through the medium to the tip of the optical fiber. Time-of-flight detectors are known and can be implemented with the optical imaging console 104 and apply optical transmission and optical reception leads within the catheter shaft 118. In a time-of-flight measurement, the optical measurement signal is emitted from the distalmost tip 124c, reflected off heart tissue along the longitudinal axis, which reflected waves or particles are received via the optical fiber, and provided to medical imaging console 104, such as an optical measurement circuit included in the medical imaging console 104. If, for instance, the distal end of the transmission fiber is pointed at septum tissue in the right atrium, the time of flight of the optical signal will be less than the time of flight of an optical signal that travels through an opening in the atrial septum and is reflected off a chamber wall in the left atrium. Accordingly, in some embodiments, a time-of-flight measurement will increase significantly or spike once the distal tip of the catheter is pointed at the opening in the septum. Also, the distalmost tip 124c of the catheter is proximate or pressed against heart tissue, the time of flight of the optical signal will be less than the time of flight of an optical signal from a catheter having a distalmost tip 124c spaced from the heart tissue. In one example, the time-of-flight measurement as a function of time can be plotted as a visualization on the display 142 to indicate a distance of the distalmost tip from the heart tissue or to detect spikes in distance indicating that the longitudinal axis is aligned with an opening in the septum.

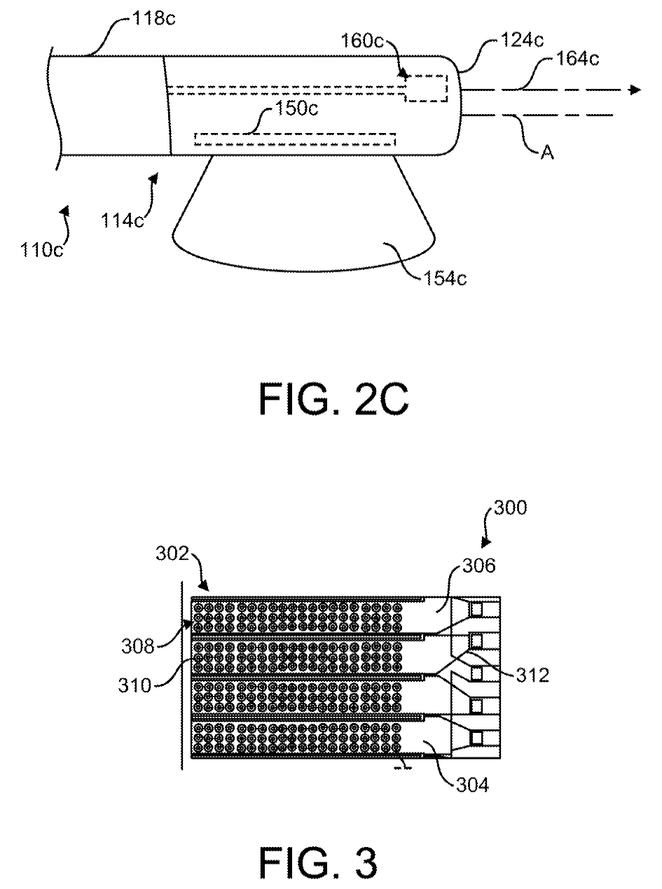

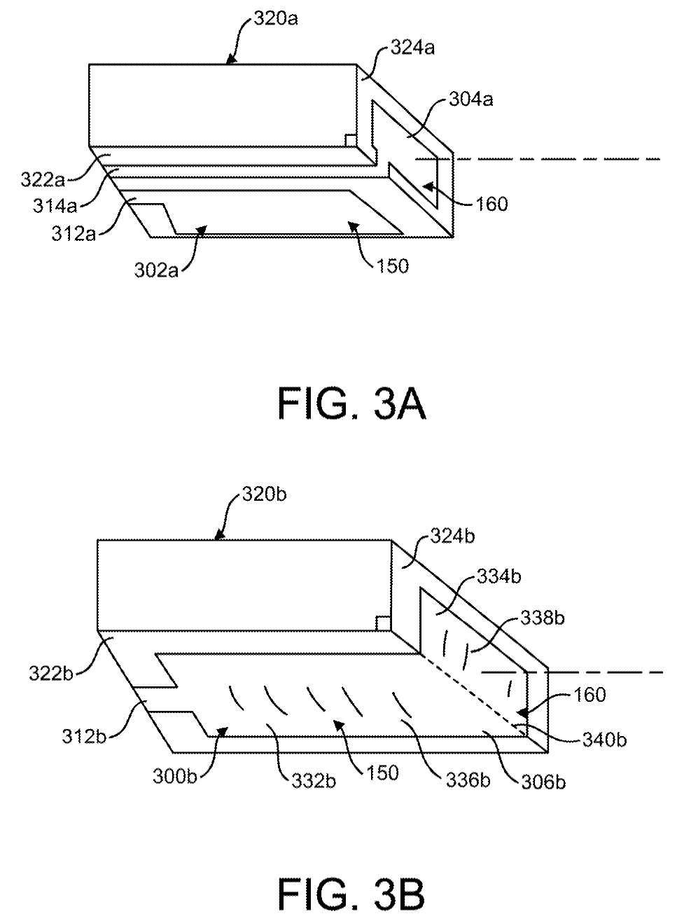

FIG. 3 illustrates an example ultrasound transducer assembly 300 that is adapted for use with the side viewing ultrasound imaging device 150 and an ultrasound transducer of the front viewing imaging device 160. The illustrated ultrasound transducer assembly 300 includes a micro-electromechanical system (MEMS) transducer array 302 such as a piezoelectric micro-machined ultrasound transducer (pMUT) or other types of MEMS transducers such as capacitive micromachined ultrasound transducers (“cMUT”), interconnected with a flexible circuit 304 for transmission and electrical interconnects. Other examples of ultrasound transducer assemblies are contemplated. In the illustrated embodiment, the flexible circuit 304 includes a flexible circuit substrate 306 with the transducer array 302, including a plurality of transducer array elements 308 comprising a plurality of transducer cells 310, and associated electrical pathways and connections 312 disposed on the flexible circuit. In the illustrated embodiment, the transducer cells 310 are pMUT cells and are configured to be arranged on the operable or active surface 152, 162. The lead conductors are connected to the ultrasound transducer assembly 300 such as via the electrical pathways and connections 312. Other examples of the ultrasound transducer assembly are contemplated, such as a transducer array and electrical pathways and connections disposed on printed circuit board. Each of the plurality of transducer array elements 308, via the lead conductors, are configured to transmit and receive, ultrasound waves. The ultrasound waves may have a bandwidth including a predetermined fundamental mode vibration of each of the plurality of transducer array elements 308, such that a single array element can transmit and receive multiple fundamental mode vibrations simultaneously. The plurality of transducer array elements 308 transmit and receive the ultrasound wave with respect to the heart or at least a portion of the heart. The transducer cells 310 are arranged in a manner to provide a wide bandwidth of the individual focused wave. In one embodiment, the ultrasound transducer assembly 300 is constructed from a pMUT array containing individual cells 310 of different diameters. In one embodiment, to achieve wider bandwidth with pMUT array elements, multiple diameters of pMUT cells are integrated into one array element. A broader bandwidth is realized through the complex interaction between the individual pMUT elements by arranging pre-shaped pMUTs with different diameters.

FIGS. 3A and 3B illustrate example arrangements of the side viewing ultrasound imaging device 150 and a front viewing ultrasound imaging device 160 in an imaging module 120 using the ultrasound transducer assembly 300. FIG. 3A illustrates an example arrangement including a plurality of ultrasound transducer assemblies 302a, 304a, each of which correspond with an example of ultrasound transducer assembly 300, such as two separate ultrasound transducer assemblies 300. The illustrated ultrasound transducer assemblies 302a, 304a are arranged perpendicular to each other, such as attached to a base member 320a having perpendicular surfaces 322a, 324a. In another embodiment, each ultrasound transducer assembly 302a, 304a can be configured on a planar circuit board and arranged perpendicular to each other. In the illustrated embodiment, the surfaces 322a, 324a are planar. In the illustrated embodiment, the base member 320a is arranged such that the ultrasound transducer assembly 302a is configured as the side viewing ultrasound imaging device 150 having planar active surface 152 configured to image in a direction perpendicular to the longitudinal axis and ultrasound transducer assembly 304a is configured as the front viewing ultrasound imaging device 160 having a planar active surface 162 configured to image in a direction along the longitudinal axis. In another embodiment, each ultrasound transducer assembly 302a, 304a can be configured on a planar circuit board and arranged perpendicular to each other such that the ultrasound transducer assembly 302a is configured as the side viewing ultrasound imaging device 150 and ultrasound transducer assembly 304a is configured as the front viewing ultrasound imaging device 160. Each ultrasound transducer assembly 302a, 304a is coupled to the lead conductors via electrical connectors 312a, 314a, respectively. In the illustrated embodiment, the ultrasound transducer assembly 304a of the front viewing ultrasound imaging device 160 is smaller in size, such as area of the operable surface, number of array elements or number of cells, than the size of the ultrasound transducer assembly 302a of the side viewing ultrasound imaging device 150. In one embodiment, the imaging module does not include multiple single side viewing ultrasound imaging devices radially spaced around the circumference of the catheter 110.

FIG. 3B illustrates an example arrangement including a single ultrasound transducer assembly 300b having a plurality of assembly segments 332b, 334b on a single flexible substrate 306b arranged perpendicular to each other, such as attached to a base member 320b having perpendicular surfaces 322b, 324b. The single ultrasound transducer assembly 330b is configured into the side viewing ultrasound imaging device 150 and the front viewing ultrasound imaging device 160. The side viewing ultrasound imaging device 150 corresponds with assembly segment 332b, and the front viewing ultrasound imaging device 160 corresponds with the assembly segment 324b. Each assembly segment 332b, 334b includes one or more transducer array elements 336b, 338b, cells and electrical pathways and interconnections 312b to create a functioning side viewing ultrasound imaging device 150 and front viewing ultrasound imaging device 160. In one embodiment, the single ultrasound transducer array assembly 300b is manufactured on a planar flexible circuit and bent, such as bent around an edge of the base member 320b, such that the assembly segments 332b, 334b are perpendicular to each other within the imaging module 120. In the illustrated embodiment, the ultrasound transducer assembly segment 334b of the front viewing ultrasound imaging device 160 is smaller in size, such as area of the operable surface, number of array elements or number of cells, than the size of the ultrasound transducer assembly segment 332b of the side viewing ultrasound imaging device 150.

The single ultrasound transducer assembly 300b of FIG. 3B includes two (or, in some embodiments, more than two) functioning or controllable ultrasound imaging devices, corresponding with assembly segments 332b, 334b, on a single flexible circuit substrate 306b. In one embodiment, a first assembly segment 332b is configurable as the side viewing ultrasound imaging device and includes a plurality of ultrasound arrays and electrical interconnections, and the second assembly segment 334b is configurable as the front viewing ultrasound imaging device and includes an ultrasound element, an ultrasound array, or a plurality of ultrasound arrays corresponding with a designed functionality. The assembly segments 332b, 334b are spaced apart on the substrate 306b to allow a bend 340b therebetween to configure the active surfaces perpendicular to each other within the imaging module 120. The controller 140 is configured to receive signals from the first assembly segment 332b to generate a first image and to receive signals from the second assembly segment 334b to generate a second image as if two separate ultrasound transducers were employed, such as in FIG. 3A.

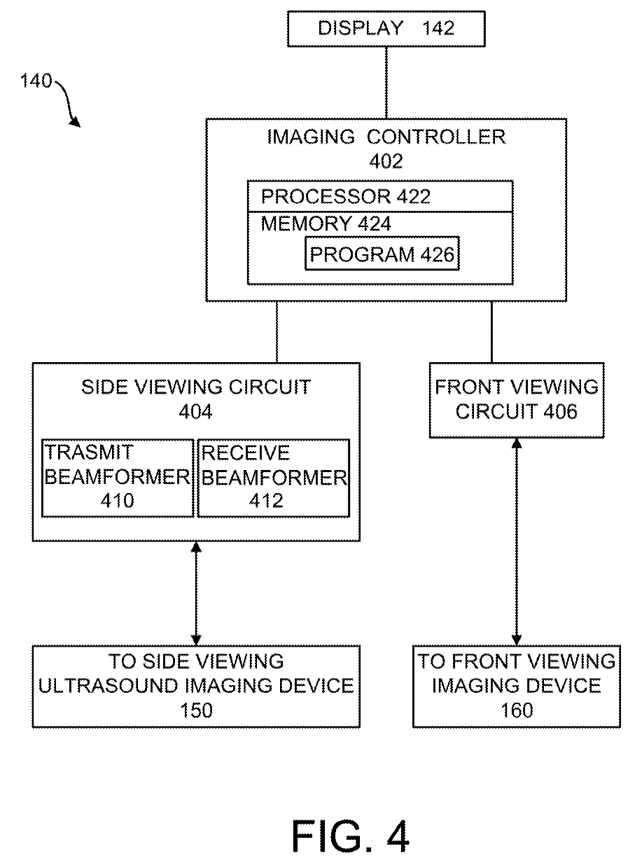

FIG. 4 illustrates an embodiment of the controller 140 of the medical imaging console 104. The controller 140 includes an imaging controller 402 coupled to side viewing circuit 404 and front viewing circuit 406. The side viewing circuit 404 is configured to be coupled to and operate with the side viewing ultrasound imaging device 150 within the medical imaging catheter 110 such as via exchanging electrical or optical signals. The front viewing circuit 406 is configured to be coupled to and operate with front viewing imaging device 160 such as via exchanging electrical or optical signals. In the illustrated embodiment, the side viewing circuit 404 includes a side viewing transmit beamformer 410 and a side viewing receive beamformer 412. In embodiments in which the front viewing imaging device 160 includes an ultrasound transducer, such as illustrated in FIGS. 2A and 2B, the front viewing circuit 406 includes a front viewing transmit beamformer and a front viewing receive beamformer. In embodiments in which the front viewing imaging device 160 includes other than an ultrasound transducer, such as an optical transducer or optical fiber as illustrated in FIG. 2C, the front viewing circuit 406 includes associated optical circuitry such as an optical transducer (if the medical imaging catheter receives an optical signal), an optical signal activator, and a time-of-flight measurement circuit.

The imaging controller 402 is implemented with any combination of hardware and programming to receive inputs from and provide outputs to side viewing circuit 404 and front viewing circuit 406, and to provide outputs to and, in some embodiments, receive inputs from the display 142. In one embodiment, the imaging controller 402 incudes a processor 422 operably coupled to a memory device 424 (a tangible storage medium). The memory device 424 can store processor-executable instructions configured to control the processor 422, such as a program 426. Examples of a memory device 424 can include a non-volatile memory device such as a read only memory (ROM), electronically programmable read only memory (EPROM), flash memory, non-volatile random-access memory (NRAM) or other memory device, and a volatile memory device such as random-access memory (RAM) or other memory device. Memory device 424 can include various combinations of one or both of non-volatile memory devices and volatile memory devices. The processor 422 includes an output port that allows the processor 422 to control the side viewing circuit 404 and front viewing circuit 406 and generate a visualization for facilitation on the display 142 according to a programmed scheme.

In other embodiments, the functionalities of imaging controller 402 are at least partially implemented in the form of electronic circuitry. Examples of electronic circuitry include integrated circuits including ASICs and programmable logic devices, such as field programmable gate arrays. A field programmable gate array is a type of integrated circuit that can be programmed or reprogrammed after manufacture and include programable logic blocks and interconnects that are configured to perform various digital functions. The logic blocks can be configured to perform combinational functions or as logic gates. Logic blocks can also include memory elements, such as flip-flops or more complete memory devices including volatile and non-volatile memory aspects that can include look up tables. Functions can be defined via a hardware description language in an electronic design automation tool to create a binary file to configure the electronic circuitry. Those skilled in the art recognize that descriptions of methods, processes, of this disclosure illustrated with the processor 422, memory 424, and program 426 can be implemented in such electronic circuitry.

The side viewing transmit beamformer 410 is configured for transmission of an electrical signal or electrical impulse towards the side viewing ultrasound imaging device 150 of the medical imaging catheter 118. The side viewing receive beamformer 412 is configured to receive an electrical signal or electrical impulse from the side viewing ultrasound imaging device 150. In embodiments in which the front viewing circuit 406 includes transmit and receive beamformers, the front viewing transmit beamformer is configured for transmission of an electrical signal or electrical impulse towards the front viewing ultrasound imaging device 160 (ultrasound transducer of the front viewing imaging device) of the medical imaging catheter 118. The front viewing receive beamformer is configured to receive an electrical signal or electrical impulse from the front viewing ultrasound imaging device 160.

The imaging controller 402 is configured to generate a visualization for facilitation on the display 142. In embodiments, the visualization includes a first section, which can occupy a first portion of a screen on the display 142 of an image obtained via the side viewing ultrasound imaging device 150. In embodiments, this first section of the visualization is a wedge-shaped image, such as a ninety-degree sector plane, which is plus/minus forty-five degrees in the axis perpendicular to the longitudinal axis A. The first section of the visualization can be presented in a variety of modes, such as brightness mode, or B-mode. The visualization also includes a second section, obtained from the front viewing imaging device, of what is in front of, i.e., in the direction of the longitudinal axis beyond the distal end, of the medical imaging catheter 110. In some embodiments, the second section of the visualization is a wedge-shaped ultrasound image, such as a ninety-degree sector plane, which is plus/minus forty-five degrees to the longitudinal axis A. In some embodiments, the second section of the visualization can be presented in a variety of modes such as B-mode. In some embodiments, the second section of the visualization is a depth or distance trace over time. In one embodiment, the first section remains displayed on the visualization during operation of medical imaging system and a clinician can selectively apply the second section of the visualization, such as when the clinician is seeking to cross into the left atrium or is visualizing the pulmonary veins. Otherwise, the clinician can turn off the second section of the visualization.

Navigation through a transseptal puncture is an example of an anticipated use of the medical imaging system 100. The left atrium is a difficult cardiac chamber to reach percutaneously. Although the left atrium can be reached via the left ventricle and mitral valve, the catheter is manipulated through two U-turns, which can be cumbersome. The transseptal puncture is a technique of creating a small passage through the atrial septum, or wall in the heart between the left and right atrium, through which a catheter can be fed. The atrial septum is punctured and dilated via tools. The transseptal puncture permits a direct route to the left atrium via the septum and systematic venous system. Increasing larger and complex medical devices can be passed into the right atrium. In the anticipated use, a clinician has punctured the septum at the fossa ovalis to create an aperture in the fossa ovalis from the right atrium to the left atrium, such as to access the pulmonary veins.

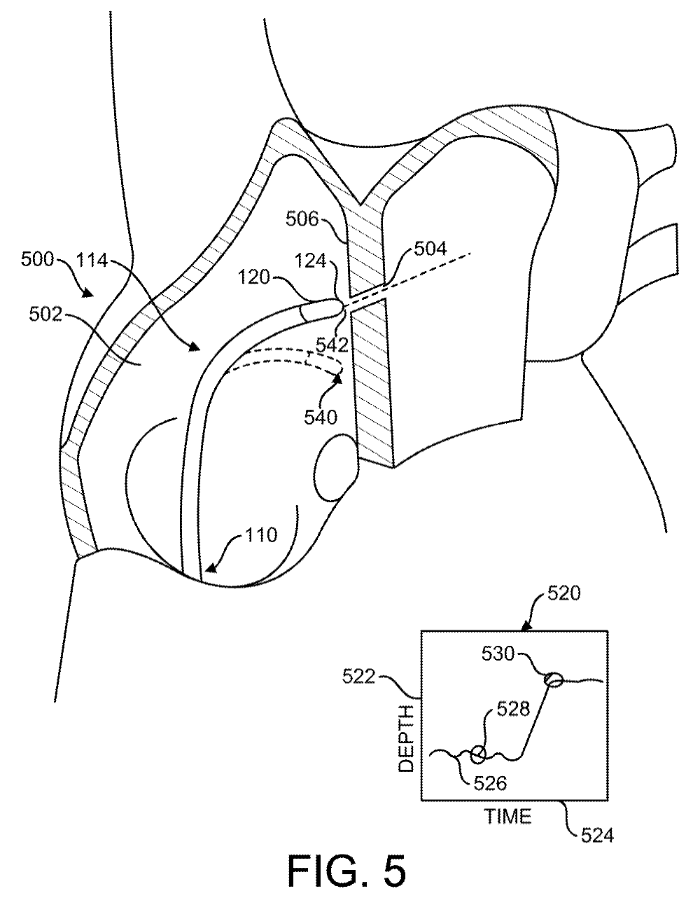

FIG. 5 illustrates the medical imaging catheter 110 after the distal region 114 has been directed through the vasculature and into the right atrium 502 of the patient’s heart 500. The image 520 from the front viewing imaging device 160 can aid the clinician to maneuver the distal tip 120 through the aperture 504 in the intra-atrial septum 506.

In one embodiment, the aperture is detected via echo return techniques, such as gauging a distance or viewing the aperture in an image generated as a function of time of a reflected acoustic signal at the ultrasound transducer. As the distal tip 124 is moved from a first position 540 (shown in phantom), in which the longitudinal axis is directed at the septum 506, to a second position 542, in which the longitudinal axis is directed at the aperture 504, the image 520, such as depth or distance 522 over time 524, can indicate the depth or distance to tissue as a trace 526. The first position 540 is indicated on the trace 526 at 528 and the second position 542 is indicated on the trace 526 at 530. The trace 526 ramps up at 530 to indicate the aperture 504. The clinician maneuvers the distal tip 124 to the aperture 504 and through the septum 506 via seeking the ramp or high distance on the trace 526. In embodiments in which the front viewing ultrasound imaging device 160 provides a planar or volume image, the clinician maneuvers the distal tip 124 toward the aperture 504 in the septum 506 as visible in the second section of the image (not shown). In each case, maneuverability is enhanced over an image from the side viewing ultrasound imaging device 150, which is pointed perpendicular to the intended direction of travel.

In another embodiment, the aperture is detected via Doppler techniques using a Doppler mode of the imaging controller 402, such as pulsed wave Doppler, continuous wave Doppler, spectral Doppler, and color Doppler. Doppler techniques apply frequency alterations that occur when ultrasound signals interact with moving objects to quantify velocity and direction of flow. There is zero to an insignificant Doppler flow detected at the first position 540, and there is measurable Doppler flow detected at the second position 542. The second section of the image can indicate Doppler flow. The clinician maneuvers the distal tip 124 to the aperture 504 and through the septum 506 via seeking the ramp or high Doppler flow on the second section of the image.

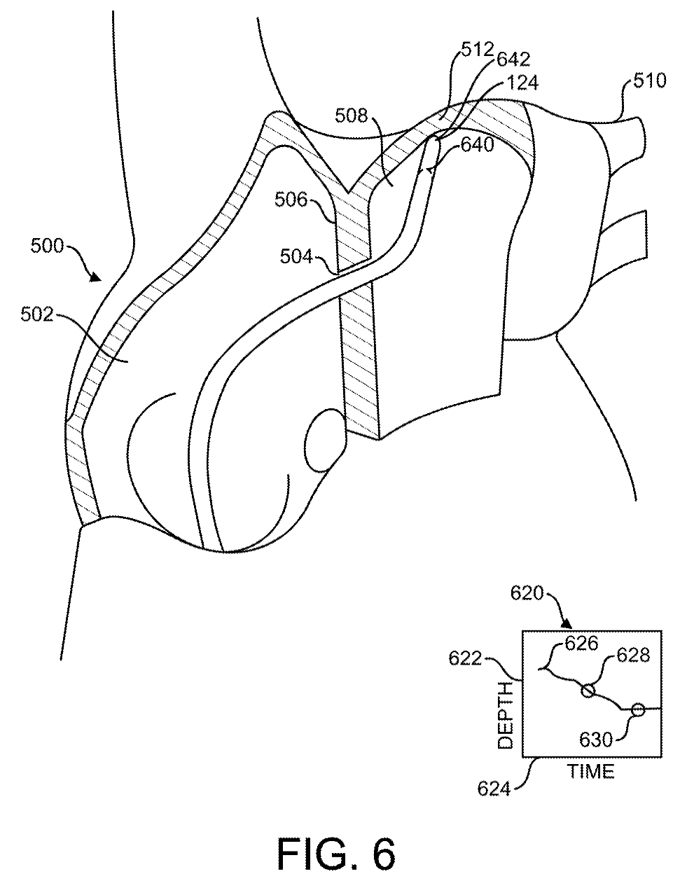

FIG. 6 illustrates the medical imaging catheter 110 after the distal region 114 has been directed through the aperture 504 and into the left atrium 508 of the patient’s heart 500. The image 620 from the front viewing imaging device 160 can aid the clinician to maneuver the distal tip 120 as the clinician views the pulmonary veins 510 with the side viewing ultrasound imaging device 150. As the distal tip 124 is moved from a first position 640 (shown in phantom), in which the distal tip 124 is relatively far from the wall 512 of the heart 500, to a second position 642, in which the distal tip 124 is relatively closer to the wall 512 of the heart 500, the image 620, such as depth or distance 622 over time 624, can indicate the depth or distance to tissue as a trace 626. The first position 640 is indicated on the trace 626 at 628 and the second position 642 is indicated on the trace 626 at 630. The depth decreases on trace 626 to indicate the distal tip 124 is close to or touching the wall 512 of the heart 500. The clinician maneuvers the distal tip 124 to in the left atrium 508 to view the pulmonary veins 512 while keeping attention on whether the distal tip 124 is pressed against the wall 512 of the heart 500. In embodiments in which the front viewing ultrasound imaging device 160 provides a planar or volume image, the clinician maneuvers the distal tip 124 as the wall 512 of the heart 500 is visible in the second section of the image, such as becoming brighter as the distal tip nears in B-mode (not shown). In each case, maneuverability is enhanced over an image from the side viewing ultrasound imaging device 150, which is pointed perpendicular to the direction of concern.

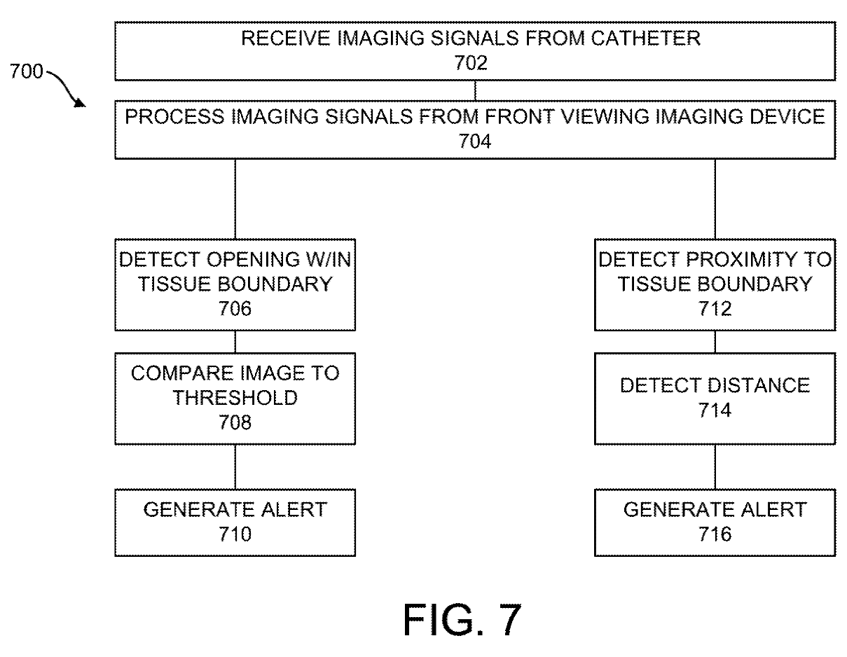

FIG. 7 illustrates an example method 700, which can be implemented with the controller 140 of the medical imaging console 104, such as via a set of executable instructions in program 426 of FIG. 4. Method 700 provides a notification or alert when an opening, such as the aperture 504 is detected and a notification or alert when the distal tip 124 is near a tissue boundary, such as the wall 512 of the heart 500. Imaging signals from the medical imaging catheter are received at 702, such as imaging signals from the side viewing ultrasound imaging device 150 and the front viewing imaging device 160. Imaging signals from the front viewing imaging device 160 are processed at 704. In one embodiment, a clinician can select between a first configuration to detect an aperture or a section configuration to detect proximity to a tissue wall. In another embodiment, the method automatically selects between the first configuration and the second configuration based on such considerations as whether the distal tip is determined to be in the right atrium or the left atrium or whether the first configuration has already been applied.

In a first configuration, the imaging signals from the front viewing imaging device 160 are processed to detect an opening with respect to a tissue boundary at 706. In one embodiment, the aperture is detected via echo return techniques. The image is processed to detect a distance above a threshold amount or ramp in distance (change in an amount of distance as a function of time) above another threshold amount. In another embodiment, the aperture is detected via Doppler techniques. The image is processed to detect an amount of flow above a threshold amount or ramp in amount of flow (change in an amount of flow as a function of time) above another threshold amount. Accordingly, an aperture is detected based on comparison of the processed image to a threshold amount or a change to another threshold amount at 708. A first alert or notification, such as an indication on a visualization in the display or a first tone on audio speaker, is provided in response to the detection of the aperture at 710.

In a second configuration, the imaging signals from the front viewing imaging device 160 are processed to detect proximity of the distal tip 124 to a tissue boundary at 712. In one embodiment, the proximity of the distal tip 124 to a tissue boundary is determined via echo return techniques. The image is processed to detect a distance below a threshold amount at 714. For example, the threshold amount can be the distal tip 124 in contact with the tissue boundary (zero distance) or the distal tip 124 very near the tissue boundary. A second alert or notification, such as a second indication on a visualization in the display or a second tone on audio speaker, is provided in response to the determination that the distal tip is at or near the tissue boundary at 716.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical imaging catheter configured to be extended into a patient’s heart, the medical imaging catheter comprising:
a shaft having a proximal region and an opposite distal region, the shaft defining a longitudinal axis; and
an imaging module coupled to the distal region and configured to be extended into the patient’s heart, the imaging module including:
a side viewing ultrasound imaging device having a planar active surface, the planar active surface configured to image in a direction perpendicular to the longitudinal axis; and
a front viewing imaging device configured to image in a direction along the longitudinal axis.

2. The medical imaging catheter of claim 1, wherein the medical imaging catheter is an intracardiac echocardiography (ICE) device.

3. The medical imaging catheter of claim 1, wherein the side viewing ultrasound imaging device includes a micro-electromechanical system (MEMS) transducer array.

4. The medical imaging catheter of claim 3, wherein the MEMS transducer array includes a piezoelectric micro-machined ultrasound transducer (pMUT).

5. The medical imaging catheter of claim 1, wherein the front viewing imaging device is configured to generate an optical signal in the direction along the longitudinal axis.

6. The medical imaging catheter of claim 5, wherein the front viewing imaging device includes an optical fiber.

7. The medical imaging catheter of claim 5, and further comprising a medical imaging console coupled to the medical imaging catheter, the medical imaging console including a controller having a time-of-flight measurement circuit configured to measure a time-of-flight of the optical signal.

8. The medical imaging catheter of claim 7, wherein the controller is configured to detect an aperture in a septum of the heart or proximity to a wall of the heart based on the measured time-of-flight.

9. The medical imaging catheter of claim 8, wherein the front viewing imaging device includes an ultrasound transducer.

10. The medical imaging catheter of claim 9, wherein the ultrasound transducer of front viewing imaging device includes a pMUT.

11. The medical imaging catheter of claim 8, and further comprising a flexible circuit substrate, wherein the side viewing ultrasound imaging device and the ultrasound transducer of the front viewing imaging device are disposed on the flexible circuit substrate.

12. The medical imaging catheter of claim 11, wherein the flexible circuit substrate includes a first planar assembly segment having the side viewing ultrasound device and a second planar assembly segment having the ultrasound transducer of the front viewing imaging device, wherein the second planar assembly segment is bent to be perpendicular to the first planar assembly segment.

13. The medical imaging catheter of claim 9, and further comprising a medical imaging console coupled to the medical imaging catheter, the medical imaging console including a controller configured to measure depth or distance of the image in the direction of the longitudinal axis and detect an aperture in a septum of the heart or proximity to a wall of the heart based on the depth of distance of the image.

14. The medical imaging catheter of claim 13, wherein the controller is configured to generate an alert if the aperture in the septum is detected or proximity to a wall of the heart is closer than a threshold amount.

15. The medical imaging catheter of claim 9, and further comprising a medical imaging console coupled to the medical imaging catheter, the medical imaging console including a controller configured to Doppler effects of the of the image in the direction of the longitudinal axis and detect an aperture in a septum of the heart based on the Doppler effects of the image.

16. An intracardiac echocardiography (ICE) catheter configured to be extended into a patient’s heart, the medical imaging catheter comprising:
a shaft having a proximal region and an opposite distal region, the shaft defining a longitudinal axis; and
an imaging module coupled to the distal region and configured to be extended into the patient’s heart, the imaging module including:
a side viewing ultrasound imaging device having a planar active surface, the planar active surface configured to image in a direction perpendicular to the longitudinal axis;
a front viewing imaging device configured to image in a direction along the longitudinal axis, wherein the front viewing imaging device includes an ultrasound transducer; and
a flexible circuit substrate, wherein the side viewing ultrasound imaging device and the ultrasound transducer of the front viewing imaging device are disposed on the flexible circuit substrate, wherein the flexible circuit substrate includes a first planar assembly segment having the side viewing ultrasound device and a second planar assembly segment having the ultrasound transducer of the front viewing imaging device, wherein the second planar assembly segment is bent to be perpendicular to the first planar assembly segment.

17. The ICE catheter of claim 16, wherein the side viewing ultrasound imaging device includes a micro-electromechanical system (MEMS) transducer array and the front viewing imaging device includes a MEMS transducer array.

18. The ICE catheter of claim 17, wherein each of the MEMS transducer arrays includes a piezoelectric micro-machined ultrasound transducer (pMUT).

19. A method for use with a medical imaging system having a medical imaging catheter configured to be extended into a patient’s heart, the medical imaging catheter comprising an imaging module coupled to a distal region of a shaft and configured to be extended into the patient’s heart, the imaging module including a side viewing ultrasound imaging device having a planar active surface, the planar active surface configured to image in a direction perpendicular to a longitudinal axis of the shaft, and a front viewing imaging device configured to image in a direction along the longitudinal axis, the method comprising at least one of:
measuring depth or distance of the image in the direction of the longitudinal axis and detecting an aperture in a septum of the heart or proximity to a wall of the heart based on the depth of distance of the image; or
applying Doppler effects of the of the image in the direction of the longitudinal axis and detecting an aperture in a septum of the heart based on the Doppler effects of the image.

20. The method of claim 19, comprising generating an alert if the aperture in the septum is detected or if proximity to a wall of the heart is closer than a threshold amount.
